(19) United States
(12) Patent Application Publication
(10) Pub. No.: US 2009/0304588 A1
(43) Pub. Date: Dec. 10, 2009

(54) BIOLOGICALLY EXCITABLE CELLS

(75) Inventor: Eduardo Marban, Beverly Hills, CA (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERSITY, Baltimore, MD (US)

(21) Appl. No.: 12/089,460

(22) PCT Filed: Oct. 16, 2006
(86) PCT No.: PCT/US2006/040228

§ 371 Date: Aug. 8, 2008

Related U.S. Application Data
(60) Provisional application No. 60/726,840, filed on Oct. 14, 2005.

(51) Int. Cl. A61K 49/00; A61K 35/12; C12N 5/00; C12N 15/87; C12N 15/00; C07K 14/47; C12N 15/11

(52) U.S. Cl. 424/9.1; 424/93.7; 424/93.21; 435/346; 435/455; 435/320.1; 530/350; 536/23.5

(57) ABSTRACT

As an alternative strategy to electronic pacemaker devices, we explored the feasibility of converting normally-quiescent ventricular myocytes into pacemakers by somatic cell fusion. The idea is to create chemically-induced fusion between myocytes and syngeneic fibroblasts engineered to express HCN1 pacemaker ion channels (HCN1 fibroblasts), in normally-quiescent myocardium. HCN1-expressing fibroblasts formed stable heterokaryons with myocytes, generating spontaneously-oscillating action potentials as well as ventricular pacemaker activity in vivo and provides a platform for an autologous, non-viral, adult somatic cell therapy. We also converted a depolarization-activated potassium-selective channel, Kv1.4, into a hyperpolarization-activated non-selective channel by site-directed mutagenesis (R447N, L448A, and R453I in S4 and G528S in the pore). Gene transfer into ventricular myocardium demonstrated the ability of this construct to induce pacemaker activity, with spontaneous action potential oscillations in adult ventricular myocytes and idioventricular rhythms by in vivo electrocardiography. Given the sparse expression of Kv1 family channels in the human ventricle, gene transfer of a synthetic pacemaker channel based on the Kv1 family has therapeutic utility as a biological alternative to electronic pacemakers.

This application claims the benefit of U.S. provisional application Ser. No. 60/726,840 filed Oct. 14, 2005, the disclosure of which is expressly incorporated herein.

### TECHNICAL FIELD OF THE INVENTION

This invention is related to the area of excitable cells. In particular, it relates to alteration of biologically excitability of cells by changing the cell's complement of ion channel proteins.

### BACKGROUND OF THE INVENTION

More than 250,000 people in the United States get artificial pacemakers implanted each year for the treatment of heart arrhythmias, typically slow or irregular heart beats. Biological pacemakers can be used to replace or augment the function of artificial pacemakers.

In the sinoatrial node, pacemaker activity is generated by a balance of depolarizing and repolarizing currents whose gating and permeation properties, in ensemble, create a stable oscillator (DiFrancesco, D. (1995) Cardiovasc Res 29, 449-56). Hyperpolarization-activated nucleotide-gated channel (HCN) family genes figure prominently in physiological automaticity, and transfer of such genes into quiescent heart tissue has been explored as one way of creating a biopacemaker (Qu, J., Plotnikov, A. N., Danilo, P., Jr, Shlapakova, I., Cohen, I. S., Robinson, R. B. & Rosen, M. R. (2003) Circulation 107, 1106-1109.; Plotnikov, A. N., Sosunov, E. A., Qu, J., Shlapakova, I. N., Anyukhovsky, E. P., Liu, L., Janse, M. J., Brink, P. R., Cohen, I. S., Robinson, R. B., Danilo, P., Jr & Rosen, M. R. (2004) Circulation 109, 506-512.; Potapova, I., Plotnikov, A., Lu, Z., Danilo, P., Jr, Valiunas, V., Qu, J., Doronin, S., Zuckerman, J., Shlapakova, I. N., Gao, J., Pan, Z., Herron, A. J., Robinson, R. B., Brink, P. R., Rosen, M. R. & Cohen, I. S. (2004) Circ Res 94, 952-959.). However, use of HCN genes may be confounded by unpredictable consequences of heteromultimerization with multiple endogenous HCN family members in the target cell (Ulens, C. & Tytgat, J. (2001) J. Biol. Chem. 276, 6069-6072.),(Brewster, A. L., Bernard, J. A., Gall, C. M. & Baram, T. Z. (2005) Neurobiology of Disease 19, 200-207.). As HCN is expressed in ventricular myocytes and may contribute to arrhythmogenesis (Cerbai, E., Pino, R., Porciatti, F., Sani, G., Toscano, M., Maccherini, M., Giunti, G. & Mugelli, A. (1997) Circulation 95, 568-571.; Hoppe, U. C., Jansen, E., Sudkamp, M. & Beuckelmann, D. J. (1998) Circulation 97, 55-65.), HCN gene transfer in vivo may have unpredicted consequences. Moreover, the use of wild-type channels offers little flexibility with regard to frequency tuning of the engineered pacemaker.

Cardiac rhythm-associated disorders are caused by malfunctions of impulse generation and conduction. Present therapies for the impulse generation span a wide array of approaches, yet remain largely palliative. Implantable devices can serve as surrogate pacemakers to sustain heart rate, or as defibrillators to treat excessively rapid rhythms. Such devices are expensive, and implantation involves a number of acute and chronic risks such as pulmonary collapse, bacterial infection, lead or generator failure (Bernstein, A. D. & Parsonnet, V. (2001) Pacing Clin Electrophysiol 24, 842-55.). The concept of cell therapy for cardiac arrhythmias differs conceptually from conventional applications. The objective here is to achieve functional re-engineering of cardiac tissue, so as to alter a specific electrical property of the tissue in a salutary manner. In this study, engineered cells are introduced to create a spontaneously-active biological pacemaker from normally-quiescent myocardium. A key ionic current present in sinoatrial nodal pacemaker cells, but largely absent in atrial and ventricular myocytes, is the pacemaker current, I_{f} (Robinson, R. B. & Siegelbaum, S. A. (2003) Annu Rev Physiol 65, 453-80.). The molecular correlates of I_{f} are hyperpolarization-activated cyclic nucleotide-gated (HCN) channels 1-4 (Stieber, J., Hofmann, F. & Ludwig, A. (2004) Trends Cardiovasc Med 14, 23-8.). We examined the use of polyethylene glycol (PEG)-induced fibroblast-myocyte fusion as a method to deliver I_{f} to myocardium and show that the heterokaryons could elicit pacemaker activity in vivo at the site of cell-injection. Because this approach is independent from cell-cell coupling and stationary to the site of fibroblast injection, it promises a stable and straightforward procedure for achieving biological pacemaker activity in a specific region of the heart.

There is a continuing need in the art for improved means of regulating cardiac rhythm malfunctions which are caused by disease, genetics, drugs, and aging, for example.

### SUMMARY OF THE INVENTION

According to one aspect of the invention method is provided for making a heterokaryon with electrical properties from both of its parent cells. An exogenous somatic cell and a fusogen reagent are injected into a site in a mammal. The exogenous somatic cell expresses an ion channel. The exogenous somatic cell fuses with an endogenous somatic cell, thereby forming a heterokaryon with electrical properties from both of its parents.

Another aspect of the invention is a method of making a biological pacemaker. Myocytes, polyethylene glycol (PEG), and syngeneic or autologous fibroblasts which express Hyperpolarization-activated cyclic-nucleotide-gated (HCN) ion channel 1 (HCN1) as shown in SEQ ID NO: 1 OR SEQ ID NO: 5 are mixed. The myocytes and the fibroblasts thereby fuse.

Yet another aspect of the invention is another method of making a biological pacemaker. An inexcitable mammalian cell is transfected with one or more nucleic acid molecules encoding a gene which depolarizes the cell membrane, a gene which repolarizes the cell membrane, and a gene which fires spontaneously. The mammalian cell thereby displays spontaneously oscillating action potentials.

One embodiment of the invention is a plasmid comprising a coding sequence for each of three ion channels. The three ion channels are HCN1 (SEQ ID NO: 1 or SEQ ID NO: 5), NaChBac (SEQ ID NO: 2), and Kir2.1 (SEQ ID NO: 3 or SEQ ID NO: 6).

Still another embodiment of the invention is a voltage-dependent K⁺ channel protein which activates upon hyperpolarization and is non-selective to monovalent cations.

Yet another embodiment of the invention is a hyperpolarization-activated, inward current, channel protein comprising four mutations relative to wild-type sequence of a Kv1.4 protein according to SEQ ID NO: 4. The four mutations are R447N, L448A, R453I, and G528S.

These and other embodiments which will be apparent to those of skill in the art upon reading the specification provide the art with tools for augmenting and repairing electrical functions in the mammalian body.

### BRIEF DESCRIPTION OF THE DRAWINGS

FIG. 1A-1E. FIG. 1A. Evidence for in vitro fusion between a guinea pig left ventricular myocyte and a fibroblast (black arrow). The fibroblasts were loaded with Calcein-AM prior to the fusion with PEG. The fusion event is evidenced by the sudden introduction of the dye from the fibroblast to the myocyte upon re-hydration. The dye is represented with orange (pseudo-colored) in green background to enhance the contrast. FIG. 1B. Spontaneously oscillating action potentials recorded from a cardiomyocyte fused with a fibroblast expressing HCN-1 channel. FIG. 1C. A representative action potential from a guinea pig fused with a control fibroblast expressing only GFP. FIG. 1D. Spontaneous action potentials recorded from an isolated myocyte fused with HCN1-fibroblast after in vivo injection. (Horizontal bar: 100 ms, vertical bar: 20 mV.) FIG. 1E. HCN1 current recorded from the fused myocyte from panel D after washing in 1 mM BaCl2.

FIG. 2A-2B. Electrocardiograms from guinea pig hearts injected with HCN1-fibroblast cells. FIG. 2A. Bipolar-pacing at 1 Hz on the site of HCN1-fibroblast injection produced ventricular beats that are the same in polarity and morphology as the ectopic ventricular beats (diagonal arrows) produced by the guinea pig's heart one day after HCN1-fibroblast injection. FIG. 2B. In some cases, junctional escape rhythms (horizontal arrows) are overtaken by ectopic ventricular beats (diagonal arrows, 16 days after cell-injection).

FIG. 3A-1 to 3B-4. Evidence of in vivo fusion between the guinea pig myocardium and HCN1-fibroblasts. FIG. 3A1-2. In vivo evidence for guinea pig myocyte-fibroblast fusion. HCN1-fibroblasts were transduced with Ad-lacZ and injected into the apex of guinea pig heart in 50% PEG1500. X-gal staining of the sections from the apex of the guinea pig heart reveals blue (X-gal) staining of longitudinal cardiomyocytes (arrows) at the border between the HCN1-fibroblasts (round blue cells) and the myocardium. FIG. 3B1-FIG. 3B4. Immunohistochemistry with a primary antibody against beta-galactosidase (green, FIG. 3B1) and myosin heavy chain (red, FIG. 3B2). The merged image (FIG. 3B-3) indicates expression of beta-galactosidases (green) in the neighboring myocytes (highlighted in a white, dotted circle) as well as in HCN1-fibroblasts transduced with Ad-lacZ (shown as a cluster of phase bright, round cells in FIG. 3B-4).

FIG. 4A-4B. Representative raw traces from HEK293 cells. FIG. 4A. Voltage-clamp recordings from HEK293 cells transfected with either NaChBac (left), hERG (middle), or Kir2.1 (right). Dotted line indicates zero current level. FIG. 4B. Action potentials from three different cells during current-clamp recordings. Each cell expresses all three channels, NaChBac, hERG, and Kir2.1. Dotted line indicates zero mV potential.

FIG. 5 A-5B. Spontaneous action potentials from HEK293 cells expressing FIG. 5A. Spontaneous action potentials from a HEK293 cell transfected with: NaChBac, HCN1, HERG, Kir2.1 (3:3:1:1, molar ratio). FIG. 5B. Spontaneous action potentials recorded from a cell transfected with single plasmid expressing NaChBac, HCN1, and Kir2.1.

FIG. 6. Design of human Kv1.4 mutations. To convert human Kv1.4 channel into “HCN-like” pacemaker channel, we focused on the S4 region as a voltage sensor and around selectivity filter region (GYG) as a determinant of ion selectivity. We speculated that the S4 triple mutations (R447N, L448A, and R453I) alter the channel's gating from depolarization-activated outward current into hyperpolarization-activated inward current and the pore mutation (G528S) of the channels render ion selectivity to nonselective for Na⁺ vs K⁺ which would induce positive shift of voltage activation.

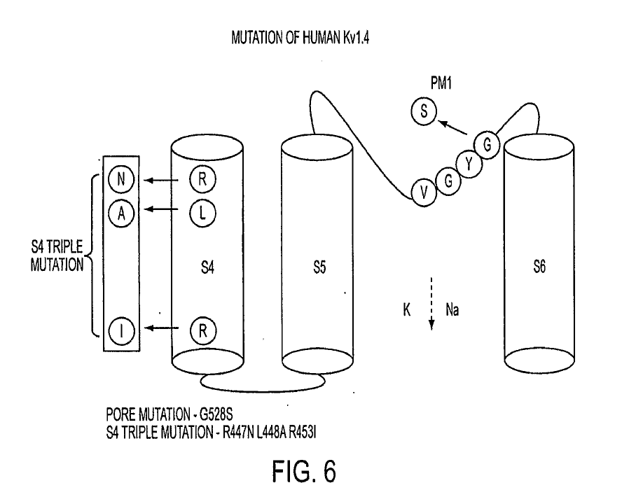

FIG. 7A-FIG. 7D. Current traces of human Kv1.4 wild type and different mutants in high K⁺ external solution. FIG. 7A. Wild-type channel showed huge depolarization-activated outward current without inward current. FIG. 7B. S4 triple mutation (_{S4T}Kv1.4) expressed substantial hyperpolarization activated inward current in high potassium solution while it hardly expressed inward current in normal Tyrode's solution (data not shown). FIG. 7C. In the pore mutant (Kv1.4_{GYS}), although current magnitude was reduced in compared with wild type, its reversal potential was changed from −80 mV (wild type) to 0 mV (data not shown). FIG. 7D. S4 triple plus pore mutation (_{S4T}Kv1.4_{GYS}) showed hyperpolarization-activated inward current in physiological conditions. This current showed time-dependent factor from −100 mV.

FIG. 8A-FIG. 8C-c. Tail-currents of _{S4T}Kv1.4_{GYS}. FIG. 8A. This channel showed very weak deactivation at potentials more negative than −80 mV. FIG. 8B. Reversal potential in normal Tyrode's was +5 mV. FIG. 8C. In high potassium (FIG. 8C-a) or equal concentration of sodium and potassium external solution (FIG. 8C-b), peak current at −150 mV was reduced by 90% or 60% in compared with the ones in normal Tyrode's, respectively. Barium did not largely affect the peak current (FIG. 8C-c) as it diminishes barium-sensitive current completely (e.g., I_{Kl}) although it likely suppressed time-dependent increasing of the current.

FIG. 9A-FIG. 9D. Effect of adeno/_{S4T}Kv1.4_{GYS} on spontaneous activity of isolated myocyte. At a holding potential of −40 mV, control isolated myocyte (FIG. 9A) expressed no measurable current, whereas _{S4T}Kv1.4_{GYS}-transduced myocyte (FIG. 9B) showed hyperpolarization-activated inward current. In this condition, mean current density was −7.2 pA/pF at −80 mV. Spontaneous action potential (AP) oscillation could be produced after first AP triggered by brief depolarizing current pulses (FIG. 9C). Raw traces showing fast spontaneous AP oscillations (FIG. 9D).

FIG. 10A-FIG. 10B-c. ECG leads II, I, III. Overview of sustained ventricular beats (FIG. 10A). Arrows indicate start and stop of sustained ventricular beats. High magnitude of same ECG (FIG. 10B-a) of dashed-line square of ECG (FIG. 10A). Junctional beats (FIG. 10B-b). Mapping of LV free wall with hand held electrode (FIG. 10B-c). Arrows indicate artifact of pacing (150 bpm).

### DETAILED DESCRIPTION OF THE INVENTION

The inventors have developed methods and products for use in biological pacemakers. In one aspect in vivo or in vitro fusion is used to improve the function of a host's endogenous excitable cells. In another aspect, an inexcitable cell is made excitable by transfer to the cell of a complement of proteins that together are sufficient to generate spontaneously oscillating action potentials. In another aspect, the inventors have developed a voltage-dependent K⁺ channel protein which activates upon hyperpolarization and is non-selective to monovalent cations.

For making fused cells, either in vitro or in vivo, any fusogen reagent known in the art can be used, whether chemical or biological. Exemplary fusogen reagents which can be used include NaNO₃, artificial sea water, lysozyme, high pH/Ca⁺⁺, polyethylene glycol (PEG), antibodies, concanavalin A, polyvinyl alcohol, dextran and dextran sulphate, fatty acids, lectins and esters. PEG of certain sizes, such as molecular weight of 500 to 2000, or 1250 to 1750, 1400 to 1600, can be advantageously used. Biological fusogens may also be used. For example, biological fusogens which can be used include Class I viral fusion proteins, e.g., HA (influenza virus hemagglutinin), Env (envelope protein for human immunodeficiency virus 1), Class II viral fusion proteins, e.g., the envelope proteins of TBE virus, intracellular vesicle fusogens, such as v-SNARE and t-SNARE, Ig domain-containing proteins such as CD9 (used during mammalian fertilization) and CD47 (for macrophage fusion), Syncytin (for trophoblast fusion in placenta). Prior to fusion, cells can be treated to make them more fusable. Such treatments may include trypsinization or other enzyme or chemical to partially degrade the cell exterior.

Heterokaryons with electrical properties from both parent cells can be made in situ, in the body of a mammal. In situ parent cells can be any cell type, such as cardiac cells, in particular cardiac myoctes, neuronal cells, striated muscle cells, endocrine secretory cells or ventricular myocytes. The in situ parent cells may not express the desired ion channel, or may not express it sufficiently or optimally. Ion channels as used herein includes transporters. Upon fusion with an exogenous somatic cell which expresses the desired ion channel the fused cell or heterokaryon acquires the ability to express the desired ion channel and gains the electrical functionality that the channel imparts. Injection of the exogenous cell can be into the heart of a mammal or other desired body location. The target host cell may be a neuronal cell. The desired channel can be a calcium channel. More specifically the desired channel can be a Hyperpolarization-activated cyclic-nucleotide-gated (HCN) ion channel 1 (HCN1). The exogenous somatic cell may be an autologous or syngeneic cell. It can be a fibroblast or any inexcitable cell, e.g., kidney cells. The exogenous somatic cell may be one that naturally expresses the desired channel, or it may be one which has acquired the ability to express the desired channel by genetic transfer of a nucleic acid which is exogenous to the exogenous somatic cell. The genetic transfer may either boost expression of the channel or provide such expression to a cell which otherwise does not express the channel. The genetic transfer may be either non-viral, for example using a plasmid, or viral, for example using adenovirus, adeno-associated virus, or lentivirus.

The fused cell or heterokaryon so formed can be used to alter excitability, for example by creating a pacemaker, alteration of cardiac repolarization, increase or decrease of muscular excitability, e.g., for the treatment of myotonic dystrophy, epilepsy, narcolepsy, memory, excitation-contraction coupling, secretion, excitation-transcription coupling.

Subsequent to administration of the exogenous cell and the fusogen, fusion and formation of a heterokaryon can be monitored by any means known in the art. These include, without limitation use of EKG and the use of immunohistochemistry for a detectable marker from the exogenous cell. Other methods for detecting ion channel activity can be used, such as patch clamp measurements.

The heterokaryons of the present invention can be made in vitro or in vivo. If made in vitro, they can be subsequently administered to mammalian body at a site in need of the electrical function of the heterokaryon.

Mammals which are amenable to the methods of the present invention include humans, rats, mice, pigs, dogs, sheep, cows, horses, etc. Any such mammal can be treated for its own sake or as an experimental model system for treating humans.

Biological pacemakers can be made from cells that are inexcitable by means of transfection (including transduction, transformation, or other means of gene transfer) with a small complement of exogenous coding sequences. As detailed below in the examples, expression of a gene which depolarizes the cell membrane, a gene which repolarizes the cell membrane, and a gene which causes a cell to fire spontaneously and repetitively is sufficient to generate oscillating action potentials in a mammalian cell which was hitherto inexcitable. The coding sequences can be delivered on one or more nucleic acid molecules or vectors. The vectors can be viral or non-viral. One particular type of inexcitable cell which can be made excitable is a human embryonic kidney cell. Examples of ion channels which can be used are HCN1 (SEQ ID NO: 1 or SEQ ID NO: 5), NaChBac (SEQ ID NO: 2), and Kir2.1 (SEQ ID NO: 3 or SEQ ID NO: 6). Others can be used as are known in the art. For example, genes which depolarize the cell membrane include those encoding a voltage-dependent sodium channel, a voltage-dependent calcium channel, and a ligand-gated cation channel such as nicotinic acetylcholine receptor. Genes which repolarize the cell membrane include those which encode a potassium channel or a chloride channel. Genes which cause a cell to fire spontaneously and repetitively include those of the HCN gene family or an engineered synthetic pacemaker channels (SPC) as described below. Such biological pacemakers can be used to for heart pacing or for treating neural or muscular disorders in which firing frequency is low, e.g., narcolepsy, Ondine's curse, or paralysis.

Also provided by the present invention is a voltage-dependent K⁺ channel protein which activates upon hyperpolarization and is non-selective to monovalent cations. One such protein is a mutant version of wild-type Kv1.4 according to SEQ ID NO: 4. The mutant version comprises four mutations relative to wild-type sequence of a Kv1.4 protein: R447N, L448A, R453I, and G528S. Other mutations having similar effects can also be used. Nucleic acids encoding coding sequences for such mutant versions of protein can be in viral or non-viral vectors, if desired. The nucleic acids can be administered to cells to form stable transfectants or transductants. The nucleic acids can also be administered to whole animals. For example, they can be delivered to a mammalian heart. In particular they can be injected into a left ventricle or atrium of a mammalian heart. They can also be delivered to neuronal sites. These mutant proteins and nucleic acids encoding them can be used as an alternative to natural pacemaker channels. These mutant proteins are more tunable and less subject to multimerization with native genes

PEG-induced membrane fusion events have served as a model system to create mouse and human hybridomas (Shirahata, S., Katakura, Y. & Teruya, K. (1998) Methods Cell Biol 57, 111-45.), to study eukaryotic cell-cell fusion events (Lentz, B. R. & Lee, J. K. (1999) Mol Membr Biol 16, 279-96.), and to deliver outward K⁺ currents into myocytes (Hoppe, U. C., Johns, D. C., Marban, E. & O'Rourke, B. (1999) Circ Res 84, 964-72.). Here, we used syngeneic fibroblasts expressing HCN1 channels as donor cells to induce spontaneous activity in normally-quiescent ventricular myocytes upon PEG-induced cell fusion. The fusion-induced biological pacemakers were stable for more than 3 weeks and functional <1 day post-injection as revealed by electrocardiography. Previous studies suggest that the fusion-induced heterokaryons can maintain the nuclei from each fusion partner separately and stably for at least several months (Gibson, A. J., Karasinski, J., Relvas, J., Moss, J., Sherratt, T. G., Strong, P. N. & Watt, D. J. (1995) J Cell Sci 108 (Pt 1), 207-14. Gussoni, E., Bennett, R. R., Muskiewicz, K. R., Meyerrose, T., Nolta, J. A., Gilgoff, I., Stein, J., Chan, Y. M., Lidov, H. G., Bonnemann, C. G., Von Moers, A., Morris, G. E., Den Dunnen, J. T., Chamberlain, J. S., Kunkel, L. M. & Weinberg, K. (2002) J Clin Invest 110, 807-14. Alvarez-Dolado, M., Pardal, R., Garcia-Verdugo, J. M., Fike, J. R., Lee, H. O., Pfeffer, K., Lois, C., Morrison, S. J. & Alvarez-Buylla, A. (2003) Nature 425, 968-73.). Stem-cell based biological pacemakers rely on cell-cell coupling for transmission of the impulse (Weimann, J. M., Johansson, C. B., Trejo, A. & Blau, H. M. (2003) Nat Cell Biol 5, 959-66.). Such gap-junctional coupling may not be stable over time; many of the major forms of human heart disease, associated with increased arrhythmic risk, coincide with gap junction remodeling and decreased cell-cell coupling (van der Velden, H. M. & Jongsma, H. J. (2002) Cardiovasc Res 54, 270-9.). Furthermore, besides the possible complications with teratoma formation, stem cells have been shown to proliferate and migrate once injected into myocardium (16.

Cao, F., Lin, S., Xie, X., Ray, P., Patel, M., Zhang, X., Drukker, M., Dylla, S. J., Connolly, A. J., Chen, X., Weissman, I. L., Gambhir, S. S. & Wu, J. C. (2006) Circulation 113, 1005-14.). This may cause unpredictable pattern of pacemaker activity from regions of heart other than the desired site. In contrast, the present approach creates biological pacemaker in situ to the site of heterokaryons formed by PEG-induced fusion. Furthermore, fibroblasts that did not undergo fusion with myocytes would not generate pacing other than the site of cell-injection due to the lack of cell-cell coupling. The present approach can be implemented with autologous, non-viral, adult cell therapy.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Materials and Methods

### Plasmid Construction, and Adenovirus Preparation, and Mutation

Human Kv1.4 cDNA was subcloned from XL-4 vector (OriGene Technologies, Inc. Rockville, Md.) to pTracerCMV2 plasmid (Invitrogen, Carlsbad, Calif.) between EcoRI and NotI sites. The adenovirus shuttle vector pAdCGI was used for generation of adeno/_{S4T}K1.4_{GYS}-IRES GFP. Adenovirus was produced as previously described¹. Oligonucleotide mutagenesis was performed with site-direct mutagenesis kit (Stratagene, La Jolla, Calif.).

### Transient Transfections of Cultured Cell Lines

HEK293 cells were seeded at a density of 2.0×10⁵ per 35-mm² the day before transfection. Cells were transfected with Lipofectamine 2000 (Invitrogen, Carlsbad, Calif.) according to manufacturer's protocol. Voltage- and current-clamp recording were carried out within 18-48 hours post-transfection.

### Ventricular Myocyte Isolation

Guinea pig left ventricular myocytes were isolated using Langendorff perfusion, as previously described². After digestion, cells were stored at room temperature in a high potassium solution (mmol/L: K-glutamate 120, KCl 25, MgCl₂ 1, glucose 10, HEPES 10, and EGTA 1; pH 7.4) for 30 minutes. For electrophysiological recordings, the cells were resuspended in normal Tyrode's (see Electrophysiology below). For in vitro fusion experiments (Section 1), myocytes were then placed on laminin-coated (20 μL/mL; Becton Dickinson, Bedford, Mass.) cover slips in 6-well plates in medium 199 (Invitrogen, Carlsbad, Calif.) supplemented with 2% FBS (Invitrogen, Carlsbad, Calif.) and maintained at 37° C. in a 5% CO₂ humidified incubator for 1 hour for fusion experiments (Section 1).

### Electrophysiology

Experiments were carried out using standard microelectrode whole-cell patch-clamp techniques³ with an Axopatch 200B amplifier (Molecular Devices Corporation, Sunnyvale, Calif.) with a sampling rate of 20 kHz and low-pass Bessel-filtered at 5 kHz. All experiments were performed at a room temperature. Cells were superfused with a Tyrode's solution containing (mmol/L) NaCl 138, KCl 5, CaCl₂ 2, glucose 10, MgCl₂ 0.5, and HEPES 10; pH 7.4. The micropipette electrode solution was composed of (mmol/L): K-glutamate 130, KCl 9, NaCl 8, MgCl₂ 0.5, HEPES 10, EGTA 2, and Mg-ATP 5; pH 7.2. Microelectrodes had tip resistances of 2 to 4 MΩ when filled with the internal recording solution. Voltage-clamp experiments were performed with an interepisode interval of 2.5 seconds. Action potentials were either initiated by short depolarizing current pulses (2 to 3 ms, 500 to 800 pA) on myocytes fused with control (GFP alone) myocytes or recorded with I=0 mode on myocytes fused with HCN1-fibroblasts. Data were corrected for the measured liquid junction potential (−18 mV)⁴ using a software JCal⁵. A xenon arc lamp was used to view Calcein-AM fluorescence or GFP at 488/530 nm (excitation/emission).

### Animal Procedure and Myocyte Isolation

Adenoviruses were injected into the left ventricular free wall of guinea pigs. Adult female guinea pigs (250-300 g) were anesthetized with 4% isoflurane, intubated, and placed on a ventilator with a vaporizer supplying 1.5-2% isoflurane. Following lateral thoracotomy, a 30-gauge needle was inserted at free wall of the left ventricle. An adenovirus of 3×10¹⁰ PFU AdSPC or 3×10¹⁰ PFU GFP (control group) was injected into the left ventricle. Forty-eight to 72 hours after injections were performed, free wall myocytes of left ventricular were isolated using standard techniques (1 Mitra R, M. M. (1986) Proc Natl Acad Sci USA. 83, 5340-4.). The yield of transduced myocytes, identifiable by their vivid green fluorescence using epifluorescence imaging, was approximately 3-5% as judged by visual assessments when cells were dispersed into the electrophysiologic recording chamber. The work presented was performed in accordance with NIH guidelines for the care and use of laboratory animals and was performed in accordance with the guidelines of the Animal Care and Use Committee of the Johns Hopkins University.

### Electrocardiograms.

Surface ECGs (BIOPAC Systems. MP100) were recorded 72 hours after adenoviral injection as previously described (Ennis, I. L., Li, R. A., Murphy, A. M., Marban, E. & Nuss, H. B. (2002) J. Clin. Invest. 109, 393-400). Guinea pigs were lightly sedated with isoflurane and needle electrodes were placed under the skin. Electrode positions were optimized to obtain maximal-amplitude recordings. ECGs were simultaneously recorded from standard limb leads I, II, and III. To detect ventricular beats effectively, we used methacholine (Sigma, 0.1-0.5 mg/g) by intraperitoneal injection to induce bradycardia. We confirm where ventricular beats originated from, by mapping LV free wall with hand held electrode.

### In Vitro Cell Fusion

The fibroblasts stably expressing HCN1 (HCN1-fibroblasts) were loaded with calcein-AM (2 μL/mL growth medium; 1 mmol/L stock solution in dimethyl sulfoxide; Molecular Probes, Eugene, Oreg.) to increase the cytosolic fluorescent marker. After staining, cells were trypsinized, centrifuged, and resuspended in 6 mL medium 199 supplemented with leukoagglutinin 40 μg/mL (Sigma-Aldrich, St. Louis, Mo.). The myocyte growth medium was exchanged with this HCN1-fibroblast suspension at 0.5 mL/well. One hour after coplating, myocytes and HCN1-fibroblasts were fused with prewarmed (37° C.) 40% polyethylene glycol 1500 (PEG) (Roche Applied Science, Indianapolis, Ind.) in H₂O. After 2 to 4 minutes of exposure to PEG, cells were rehydrated with high potassium solution (same solution that was used after myocyte isolation) for 5 to 10 minutes and then superfused with normal Tyrode's solution (see below).

### Recombinant Lentivirus Production to Create a Stable Fibroblast Cell Line Expressing HCN1

Recombinant lentiviruses were generated by the 3-plasmid system⁶ by co-transfecting HEK293 cells with pLentiV-CAG-HCN1-IRES-GFP, pMD.G, and pCMVΔR8.91. The lentiviral construct expresses the pacemaker channel, HCN1, under the composite promoter CAG, and then expresses green fluorescent protein (GFP) after internal ribosomal entry site (IRES). Guinea pig lung fibroblasts (ATCC, Manassas, Va.) were grown to 80% confluency in 75 cm² flasks in F12K media supplemented with 10% FBS (Invitrogen, Carlsbad, Calif.). The fibroblasts were stably transduced with pLentiV-CAG-HCN1-IRES-GFP at a final concentration of 10,000 TU/mL with 8 μg/mL polybrene to facilitate transduction. The HCN1-GFP transduced fibroblasts were selected using fluorescence activated cell sorting (FACS). Flow cytometry was performed using a Facstar (Becton Dickinson, Bedford, Mass.) and analyzed using CellQuest (Becton Dickinson, Bedford, Mass.). Non-transduced guinea pig lung fibroblasts were used as non-fluorescent controls. Green fluorescent protein (GFP)-positive cells were measured as those whose fluorescence intensity exceeded the fluorescence of 99.9% of the control cells (488/530 nm excitation/emission).

Adenovirus Transduction of HCN1-Fibroblasts and Cell Injection into Guinea Pig Heart

The E. coli β-galactosidase encoded by lacZ gene was subcloned into an adenoviral shuttle vector pAd-Lox to generate pAd-Lox-LacZ by Cre-Lox recombination in Cre-4/HEK293 cells as described⁷. HCN1-fibroblasts were transduced with Ad-lacZ for 6 hours prior to injection into a guinea pig heart. Adult female guinea pigs (250-300 g) were anesthetized with 4% isoflurane, intubated, and placed on a ventilator with a vaporizer supplying 1.5-2% isoflurane. Typically 1×10⁵ HCN1-fibroblast cells were trypsinized (0.05%), resuspended in 100 mL of 50% PEG 1500, and injected intramyocardially at the apex of a guinea pig heart with a 30G1/2 needle.

For the adenoviral injection of synthetic pacemaker ion channels (Section 3), the virus solution of 3×10¹⁰ PFU Ad/_{S4T}K1.4_{GYS} or 3×10¹⁰ PFU GFP (control group) was injected into the left ventricle. Forty-eight to 72 hours post-injection, free wall myocytes of left ventricular were isolated using standard techniques⁸. The yield of transduced myocytes, identifiable by their vivid green fluorescence using epifluorescence imaging, was approximately 3-5% as judged by visual assessments.

The work presented was performed in accordance with NIH guidelines for the care and use of laboratory animals and was performed in accordance with the guidelines of the Animal Care and Use Committee of the Johns Hopkins University.

### Electrocardiograms

Surface ECGs were recorded using MP100 (BIOPAC Systems. Goleta, Calif.) between 1-16 days after the fibroblast injection (Section 1) or 72 hours after adenoviral injection (Section 3) as previously described⁹. ECGs were simultaneously recorded from standard limb leads I and III after the guinea pigs had been sedated with 1.8% isoflurane using a 2-lead digital ECG system at 2 kHz (Lead 1 and Lead 3, BIOPAC Systems, Goleta, Calif.). Lead 2 was off-line calculated by Einthovan's triangle using Acqknowlege 3.7.3 software (BIOPAC Systems, Goleta, Calif.). In order to unleash the ectopic ventricular beats originated from the injection sites of the biological pacemakers (Section 1 and 3), we performed peritoneal injection of methacholine (0.1-0.5 mg per kg of body weight in saline, Sigma-Aldrich, St. Louis, Mo.), thus slowing the heart rate.

### Cell Fusion and Dye Loading

The fibroblasts stably expressing HCN1 (HCN1-fibroblasts) were loaded with Calcein-AM (2 μL/mL growth medium; 1 mmol/L stock solution in dimethyl sulfoxide; Molecular Probes, Eugene, Oreg.) to increase the cytosolic fluorescent marker. After staining, cells were trypsinized, centrifuged, and resuspended in 6 mL medium 199 supplemented with leukoagglutinin 40 μg/mL (Sigma-Aldrich, St. Louis, Mo.). The myocyte growth medium was exchanged with this HCN1-fibroblast suspension at 0.5 mL/well. One hour after co-plating, myocytes and HCN1-fibroblasts were fused with pre-warmed (37° C.) 40% polyethylene glycol 1500 (PEG) (Roche Applied Science, Indianapolis, Ind.) in H₂O. After 2 to 4 minutes of exposure to PEG, cells were re-hydrated with high potassium solution (same solution that was used after myocyte isolation) for 5 to 10 minutes and then washed with normal Tyrode's solution (see below).

### Electrophysiology

Experiments were carried out using standard microelectrode whole-cell patch-clamp techniques (Hamill, O. P., Marty, A., Neher, E., Sakmann, B. & Sigworth, F. J. (1981) Pflugers Arch 391, 85-100.) with an Axopatch 200B amplifier (Axon instruments) with a sampling rate of 20 kHz and low-pass Bessel-filtered at 5 kHz. All experiments were performed at a room temperature. Cells were washed with a normal Tyrode's solution containing (mmol/L) NaCl 138, KCl 5, CaCl₂ 2, glucose 10, MgCl₂ 0.5, and HEPES 10; pH 7.4. The micropipette electrode solution was composed of (mmol/L): K-glutamate 130, KCl 9, NaCl 8, MgCl₂ 0.5, HEPES 10, EGTA 2, and Mg-ATP 5; pH 7.2. Microelectrodes had tip resistances of 2 to 4 MΩ when filled with the internal recording solution. Voltage-clamp experiments were performed with an inter-episode interval of 2.5 seconds. Action potentials were either initiated by short depolarizing current pulses (2 to 3 ms, 500 to 800 pA) on myocytes fused with control (GFP alone) myocytes or recorded with I=0 mode on myocytes fused with HCN1-fibroblasts. Data were corrected for the measured liquid junction potential (−18 mV)(Neher, E. (1992) Methods Enzymol 207, 123-31.). A xenon arc lamp was used to view Calcein-AM fluorescence or GFP at 488/530 nm (excitation/emission).

### X-Gal Staining and Immunohistochemistry:

Guinea pig hearts were excised and frozen-sectioned in OCT (VWR Scientific, West Chester, Pa.) 5 μm slices. Alternating sections were used for either immunohistochemistry or staining with 5-bromo-4-chloro-3-indolyl-β-D-galactoside (X-gal). The sections were fixed in 2% formaldehyde-0.2% glutaraldehyde for 15 min at room temperature, and stained for 6 h at 37° C. in PBS containing 1.0 mg/ml X-gal, 15 mM potassium ferricyanide, 15 mM potassium ferrocyanide and 1 mM MgCl2. After staining, the slices were washed with PBS twice. For immunohistochemistry, 200-fold diluted rabbit polyclonal against β-galactosidase (FITC-conjugated, Abcam, Cambridge, Mass.) and 400-fold diluted mouse cardiac myosin heavy chain (MHC, Abcam, Cambridge, Mass.) were used for primary antibodies and AlexaFluor588 anti-mouse (diluted 200-fold, Invitrogen, Carlsbad, Calif.) was used for secondary antibody against cardiac MHC. The sections were blocked with 10% goat serum+0.01% TritonX-100 in PBS before the primary and secondary antibody incubation. All antibodies were diluted in 2% goat serum+0.01% TritonX-100 and incubated on the sections for 45 min at room temperature.

### Example 2

### Creation of a Biological Pacemaker by Cell Fusion

As an alternative strategy to electronic pacemakers or to gene therapy/stem cell approaches, we explored the feasibility of converting ventricular myocytes into pacemakers by cell fusion. The idea is to create chemically-induced fusion between ventricular myocytes and syngeneic fibroblasts engineered to express pacemaker ion channels, HCN1.

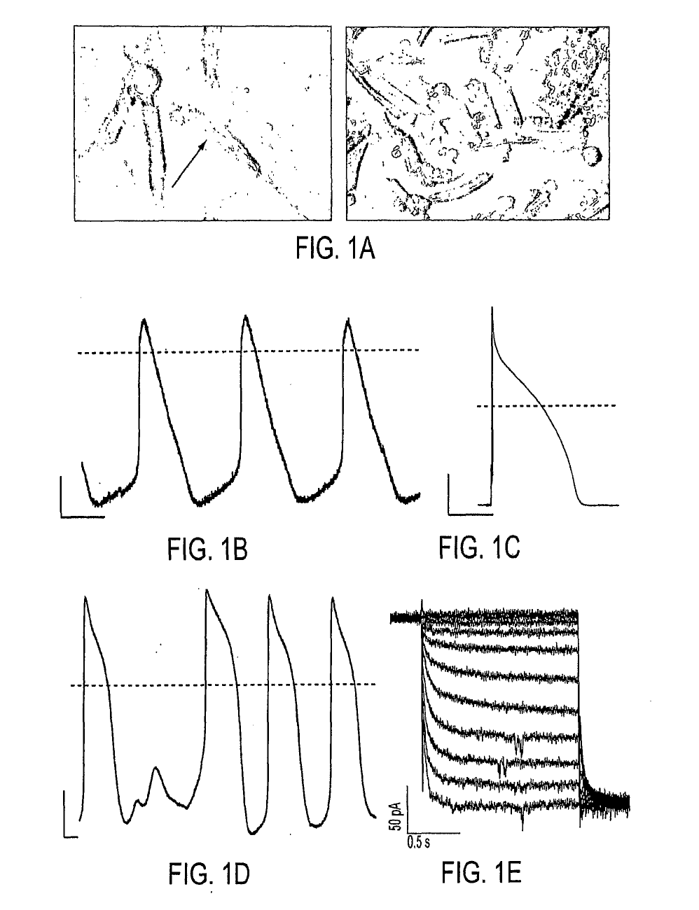

In order to examine fusion events, guinea pig lung fibroblasts stably expressing HCN1 channels (HCN1-fibroblasts) were fused with freshly-isolated guinea pig ventricular myocytes using polyethylene glycol (PEG). Within 3 minutes of dehydration and rehydration, the HCN1-fibroblasts fused with ventricular myocytes as verified by the sudden introduction of Calcein-AM fluorescence into the myocytes (FIG. 1A). Current-clamp of the myocyte/HCN1-fibroblast heterokaryon exhibited spontaneous action potentials with a slow phase-4 depolarization (FIG. 1B), suggesting the expression of pacemaker current, I_{f}. The spontaneous pacemaker activity was not observed in myocytes fused with control fibroblasts expressing GFP only (FIG. 1C).

The maximum diastolic potentials of the heterokaryons formed with HCN1-fibroblasts were only modestly depolarized (−76±9 mV, n=9) relative to the resting membrane potentials of the heterokaryons formed with control fibroblasts (−80.5±2 mV, n=7). Subsequent voltage-clamp recordings with 1 mM external Ba²⁺ to block I_{Kl} revealed the heterologously-expressed pacemaker current, I_{f}, which was not detectable either in ventricular myocytes alone or in myocytes fused with control fibroblasts. Freshly-isolated heterokaryons formed by in vivo fusion between myocytes and HCN1-fibroblasts expressed robust pacemaker current with a conductance of −770±7 pS/pF (n=9, FIG. 1D), an I_{f} density >2-fold that reported in isolated rabbit sinoatrial nodal cells (Honjo, H., Boyett, M. R., Kodama, I. & Toyama, J. (1996) J Physiol 496 (Pt 3), 795-808; van Ginneken, A. C. & Giles, W. (1991) J Physiol 434, 57-83.). The I_{f} expressed from heterokaryons exhibited normal HCN1 activation kinetics with a potential of half-maximal activation of −73.1±2.2 mV. The chemically-induced in vivo fusion events did not alter the main excitatory ionic current, I_{Na}, of the heterokaryons (FIG. 1F; 22.1±3 nA [n=9] at −40 mV for myocytes fused with HCN1-fibroblasts vs. 20.8±3 nA [n=7] for GFP-alone control fibroblasts). Cell fusion should be accompanied by an increase in total cell surface area, a parameter which can be indexed by measurements of electrical capacitance. Indeed, GFP-positive heterokaryons exhibited a larger membrane capacitance than the GFP-negative myocytes (124±14 pF, n=9 and 97±8 pF, n=15, respectively, p<0.05), supporting the concept of in vivo fusion events. The increased cell capacitance, in effect, would dilute the density of hyperpolarizing-current, I_{Kl} by 20%. Thus, the robust I_{f} conductance combined with the decreased I_{Kl} conductance drives the spontaneous pacemaking in the heterokaryons.

Equipped with these data, we focally-injected the HCN1-fibroblasts suspended in 50% PEG into the apex of a guinea pig heart. Langendorff-isolation of ventricular myocytes from the site of HCN1-fibroblast injection revealed GFP-positive myocytes which exhibited spontaneous pacemaker activity with a gradual phase 4-depolarization (FIG. 1D). Indeed, subsequent voltage-clamp recordings with 2 mM external Ba²⁺ to block I_{Kl} revealed the heterologously expressed I_{f}, which was not detectable either in ventricular myocytes alone or in myocytes fused with control fibroblasts expressing only GFP (FIG. 1E).

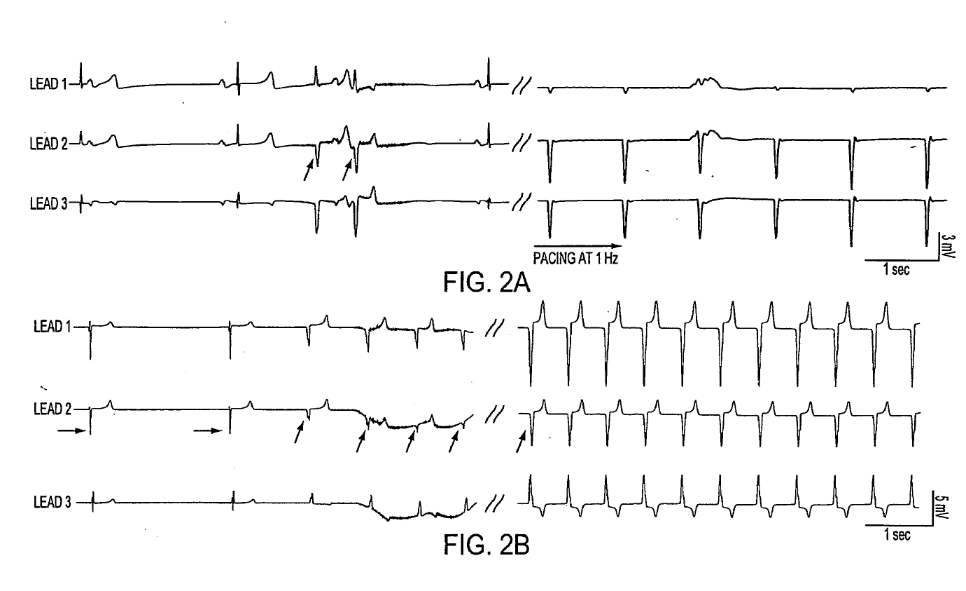

In order to examine ectopic pacemaker activity generated by the in vivo fusion, guinea pigs' heart rates were slowed with methacholine injection. Electrocardiograms recorded 1-16 days after the HCN1-fibroblast-injection revealed ectopic ventricular beats that were identical in polarity and similar in morphology to those recorded during bipolar pace-mapping of the apex in the same animal (FIG. 2A, n=5 of 13). Occasionally, junctional escape rhythms (horizontal arrows) could be overtaken by ectopic ventricular pacemaker activity (FIG. 2B). These ectopic beats were not observed in animals injected with control fibroblasts expressing GFP only (data not shown, n=4).

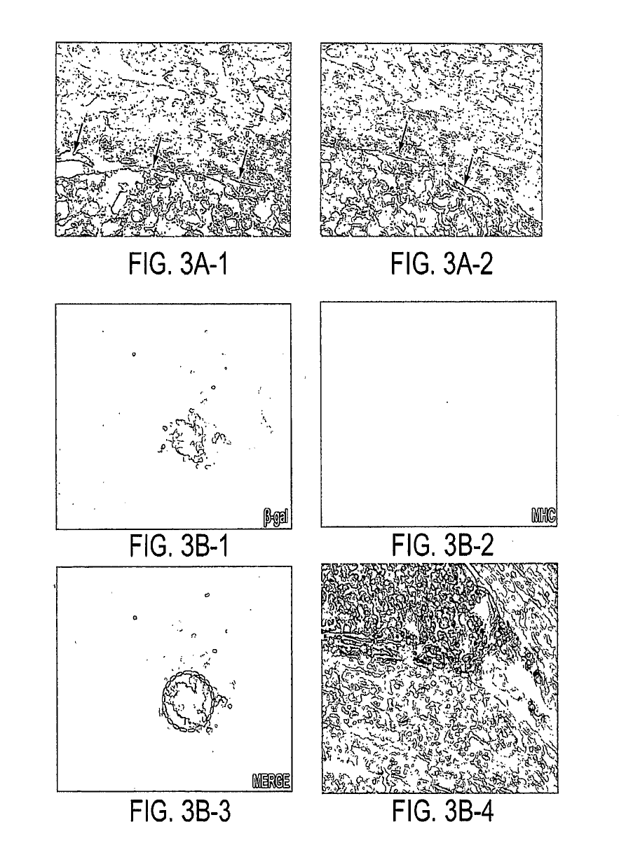

To investigate in vivo fusion events, the HCN1-fibroblasts were transduced with adenovirus expressing β-galactosidases encoded by lacZ gene (Ad-lacZ). X-gal staining of the heart sections at the site of cell-injection revealed the presence of β-galactosidases in the longitudinal ventricular myocytes at the border of myocytes and HCN1-fibroblasts as well as in the HCN1-fibroblasts that did not undergo fusion with myocytes (FIG. 3A). Immunohistochemistry against β-galactosidase and myosin heavy chain (MHC) co-localized the two proteins on cardiomyocytes (FIG. 3 B-FIG. 3E), suggesting that the β-galactosidases from the HCN1-fibroblasts' cytoplasm mixed into cardiomyocytes' cytoplasm upon cell fusion.

One could speculate that the I_{f} from HCN1-fibroblasts was relayed to cardiomyocytes by cell-cell communication between fibroblasts and myocytes. To examine the possibility of cell-cell coupling, a population of HCN1-fibroblasts were loaded with a membrane impermeable dye Calcein-AM and mixed with un-loaded HCN1-fibroblasts. The dye did not diffuse from a loaded-HCN1-fibroblast to the neighboring HCN1-fibroblast indicating no cell-cell coupling mechanism in these fibroblasts (data not shown). These data suggest that the pacemaker activity instructed by I_{f} has likely been generated from the fused heterokaryons between myocytes and HCN1-fibroblasts exclusively rather than electronic coupling between myocytes and fibroblasts. Taken together, these data provide strong evidences for a biological pacemaker activity originated from the heterokaryons upon chemically-induced cell fusion between ventricular myocytes and HCN1-fibroblasts.

PEG-induced membrane fusion events have served as a model system to create mouse and human hybridomas¹⁰, study the eukaryotic cell-cell fusion events¹¹, and been used to rapidly introduce transient outward K⁺ currents into guinea pig ventricular myocytes, thereby modifying guinea pig action potential profile². Here, we used syngeneic fibroblasts expressing HCN1 channels as donor cells in order to impart phase 4-depolarization in guinea pig ventricular myocytes upon PEG-induced cell fusion. The fusion-induced biological pacemakers are functional as early as 1 day post-injection and stable for at least more than 2 weeks. Previous studies suggest that the fusion-induced heterokaryons can maintain the nuclei from each fusion partner separately and stably over at least several months¹²⁻¹⁵. Our approach capitalizes on the immediateness and the stableness of these heterokaryon-pacemakers induced by generally inert chemical, PEG. Furthermore, unlike previous biological pacemakers¹⁶, the present approach is not dependent on cell-cell coupling and can be implemented with autologous, non-viral, adult cell therapy.

### Example 3

### Conversion of Non-Excitable Cells to Self-Contained Biological Pacemakers

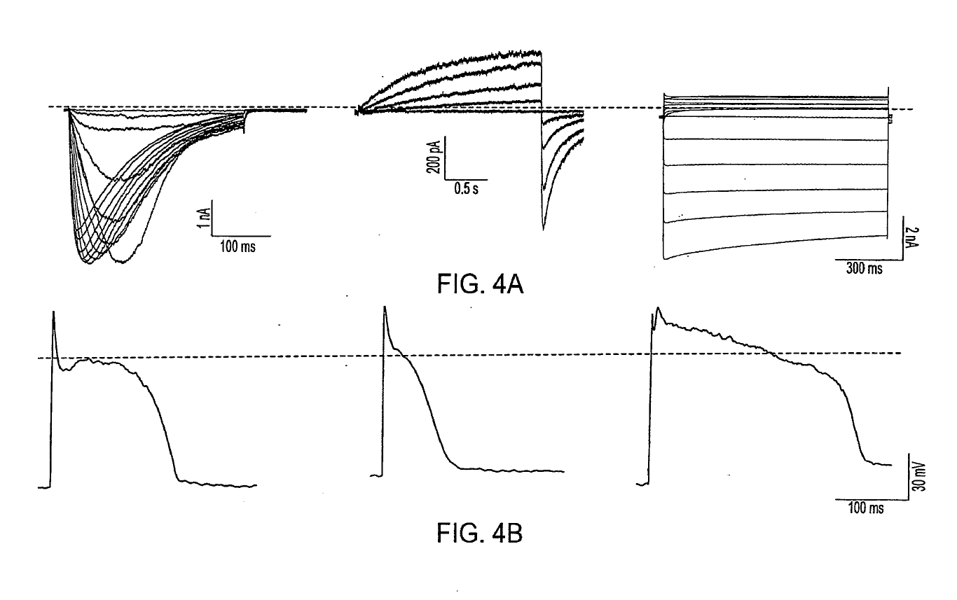

In pacemaker cells of the sinoatrial node, voltage- and time-dependent membrane ionic currents generate spontaneous action potentials (APs). We hypothesized that a non-excitable cell could be converted into a pacemaker by heterologous expression of a minimal complement of specific ion channels. To this end, HEK293 cells were engineered to express the following ionic currents: 1) an excitatory current 2) an early repolarizing current, and 3) an inward rectifier current. A Na⁺ channel from bacteria (NaChBac)¹⁷ (FIG. 4A, left) was chosen for the excitatory current because of its slow gating kinetics and its compact cDNA, human ether-a-go-go related gene channels (hERG)¹⁸ (FIG. 4A, middle) for repolarizing current to activate and counter the depolarizing effects of NaChBac, and Kir2.1¹⁹ (FIG. 4A, right) to favor a negative diastolic potential.

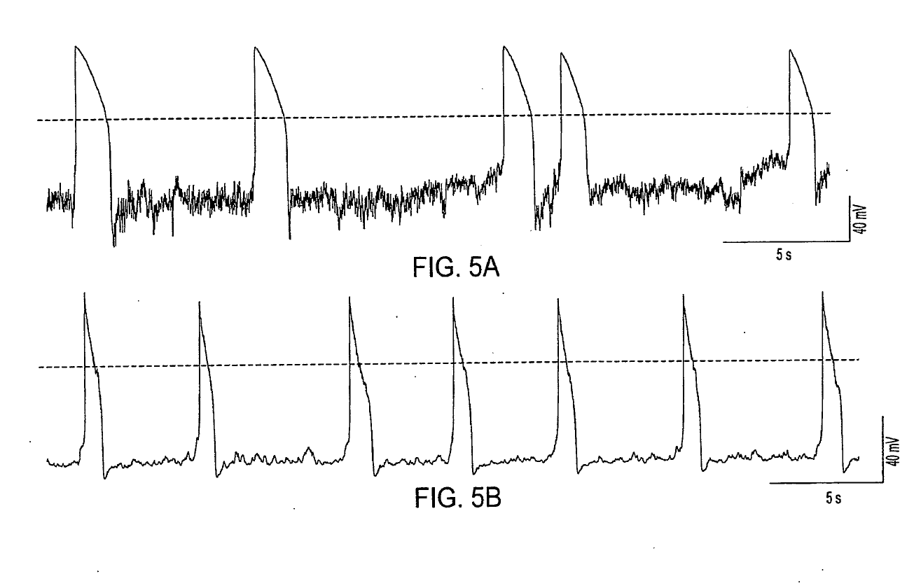

In current-clamp recordings at room temperature, action potentials could be generated from HEK cells expressing all three ion channels (n=5/31) upon stimulation with brief depolarizing currents (0.3 to 0.7 nA) (FIG. 4B). The maximum diastolic potentials (MDP) were −78±7 mV with an AP duration at 90% repolarization (APD₉₀) value of 575±33 ms (n=5). Mathematical modeling based on the Luo-Rudy guinea-pig formulation suggested that addition of I_{f}, in addition to I_{Kl}, I_{Na}, and Iₜₒ, could trigger the myocyte to beat spontaneously²⁰. Equipped with these data, HCN1 was further co-expressed to provide I_{f}, a hyperpolarization-activated depolarizing current. Whole-cell recordings from the quadruple-transfected HEK cells revealed spontaneous APs resembling the AP morphology of ventricular myocytes but with slow phase-4 depolarizations, a hallmark of native cardiac pacemaker cells (FIG. 5A). The spontaneous APs exhibited an MDP of −81.5+11.8 mV, maximum rate of rise (dV/dtₘₐₓ) of 21.6±8.6 V/s, APD₉₀ of 660±189 ms, and frequency of 3±1 bpm (n=4).

In an effort to package all necessary channel genes in a single plasmid, HCN1, NaChBac, Kir2.1-GFP were subcloned in tandem via IRES to yield a triple-gene construct. The idea was to create single plasmid that could generate spontaneously oscillating action potentials in HEK293 cells. The hERG channel was omitted after recognizing that most HEK293 cells express endogenous outward K⁺ currents (data not shown), which could counter the depolarizing effect of I_{Na}. Expectedly, current-clamp recordings of some of the triple-gene-transfected HEK293 cells exhibited spontaneously oscillating action potentials (FIG. 5B). Taken together, the present data determined the essential and sufficient set of ion channels for pacing and demonstrate the creation of the first self-contained biological pacemaker in non-excitable human cells.

### Example 4

### Synthetic Pacemaker Channels

Pacemaker activity is the product of a balance between depolarizing currents and repolarizing currents whose gating and permeation properties, in ensemble, create a stable oscillator. One key element of nodal pacemakers is the pacemaker current encoded by the HCN channel gene family. While HCN channel gene transfer has been used to engineer biological pacemakers²¹, this strategy may be confounded by unpredictable consequences of heteromultimerization with multiple endogenous HCN family members in the target cell^{22,23}. Moreover, the use of wild-type channels offers little flexibility with regard to frequency tuning of the engineered pacemaker. Here, by selective mutagenesis involving <2% of the coding sequence, we have converted a depolarization-activated K⁺-selective channel, Kv1.4, into a hyperpolarization-activated inward current.

### _{S4T}Kv1.4_{GYS} Expresses Hyperpolarization-Activated Inward Current in Physiological Condition.

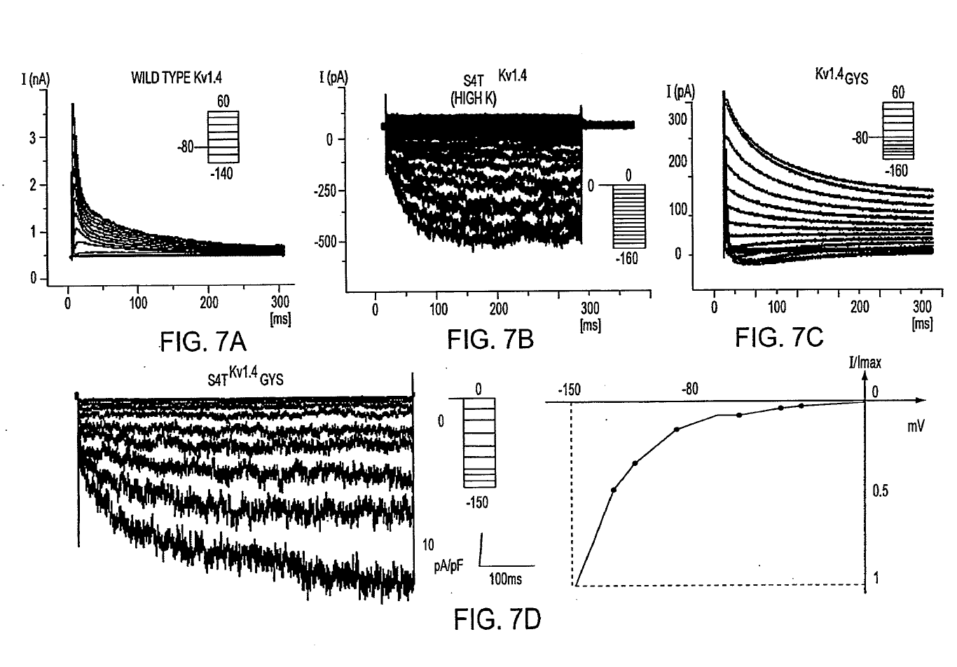

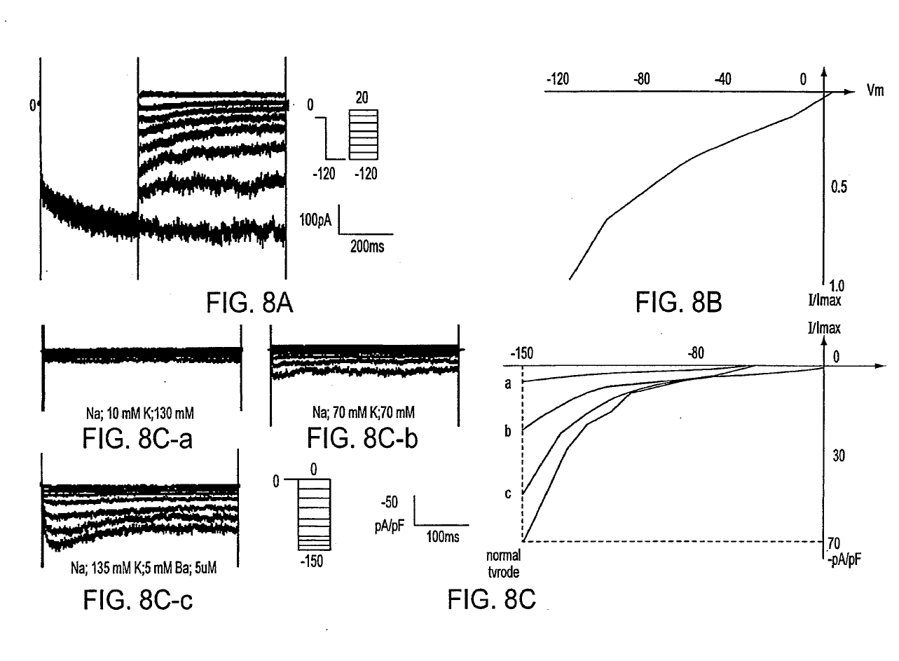

We first sought to alter the gating of Kv1.4 so as to render the channels hyperpolarization-activated. Based on a previous report²⁴, we designed a channel expressing hyperpolarization-activated inward currents (similar to HCN channels) in Kv1.4 channels under physiological conditions. In the Kv1.4 backbone, we introduced three point mutations (R447N, L448A, and R453I) in the S4 segment and a single mutation (G528S) in the pore (FIG. 1)(Heginbotham L, M. R. (1993) Biophys J. 65, 2089-96.; Miller A G, A. R. (1996) Neuron 16, 853-8.). Triple mutations in the S4 region, R447N, L448A, and R4531 (_{S4T}Kv1.4) showed hyperpolarization activated inward currents in high K⁺ external solution when expressed in HEK293 cells (FIG. 7B), but its reversal potential was still −80 mV (data not shown). In order to make positive shift of voltage activation, we further mutated the pore region to render the channels nonselective for Na⁺ vs K⁺ based on the previous studies on ion selectivity in K⁺ channels²⁵. By mutating a residue (G528S) in the selectivity filter of the Kv1.4 channel pore, the Kv1.4_{GYS} mutant channels expressed depolarization-activated small outward current (almost one-tenth of wild type Kv1.4) with tiny inward current in negative voltage range (FIG. 7C). Combining the S4 triple and pore mutations within a Kv1.4 channel, _{S4T}Kv1.4_{GYS} channels expressed hyperpolarization-activated inward currents in physiological condition (FIG. 7D). Mean current densities of _{S4T}Kv1.4_{GYS} at −130 mV was −30.3 pA/pF mV (n=10). Tail current voltage relationship indicated that the reversal potential was around 0 mV, and deactivation was very weak and mostly absent at −100 mV (FIGS. 8A and B). Taken together, _{S4T}Kv1.4_{GYS} channels express large hyperpolarization-activated inward currents in the physiological condition with no inactivation and very weak deactivation at potentials more negative than −80 mV. We further investigated how the outer bath solution could affect the _{S4T}Kv1.4_{GYS} currents using high potassium (K; 130 mM, Na; 10 mM), equal concentration of sodium and potassium (Na; 70 mM K; 70 mM), and normal Tyrode's with barium (Na; 135 mM K; 5 mM Ba; 5 uM) as external solutions. In high potassium solution (FIG. 8C-a), maximal current density was drastically reduced in comparison with control normal Tyrode's (data not shown, refer to FIGS. 7D, E) while it was hardly affected in normal Tyrode's with barium (FIG. 8C-c). In equal concentration of sodium and potassium (FIG. 8C-b) also, it was reduced by 60%. These results confirmed that _{S4T}Kv1.4_{GYS} is a non-selective channel with high permeability of sodium and its current is not sensitive to barium. Potassium per sodium permeability ratio (P_{Na}/P_{K}) was calculated to be 1.08 by Goldman-Hodgkin formula (n=5). In the light of the fact that the Kv1.4 channels do not form hetero-multimers with HCN-channels²³, these _{S4T}Kv1.4_{GYS} channels could function as synthetic pacemaker ion channels in the absence of HCN-channels.

Action Potential Oscillation was Detected in Isolated Myocyte Transduced with Adeno/_{S4T}Kv1.4_{GYS}.

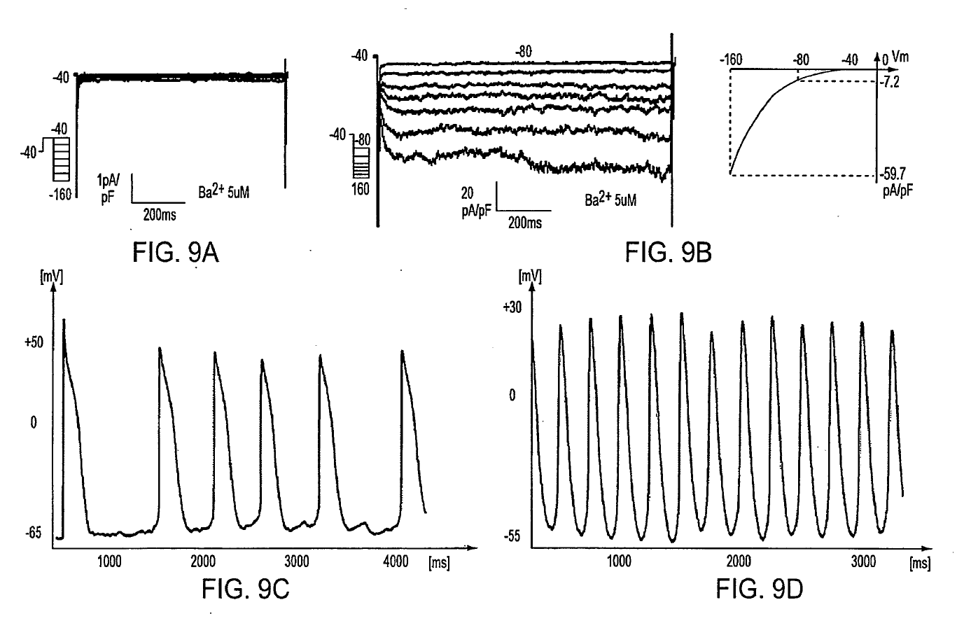

We isolated guinea-pig myocyte 72 hours after injection of Adeno/_{S4T}Kv1.4_{GYS} and patched GFP-positive cells. There was little measurable pacemaker current in control cells from injected animals (FIG. 9A). In contrast, we detected hyperpolarization-activated inward current of _{S4T}Kv1.4_{GYS} channel (FIG. 9B), although external barium might modify the phenotypes of this current partially. Under this condition, mean current density at −80 mV or −160 mV was −7.2 pA/pF or −59.7 pA/pF mV, respectively (n=6 each). We also examined action potential (AP) of control GFP-negative (n=13) and GFP-positive cells (n=14). There was no significant difference in evoked-action potential durations (306.2 ms:control versus 303.2 ms: GFP positive). Control cells never exhibited spontaneous AP oscillation, whereas half of GFP positive cells exhibited spontaneous AP oscillation (FIG. 9C) although this oscillation continued only for a short time (usually less than 10 sec). Sometimes, we also detected fast rhythm of AP (mean rate was more than 200 bpm, FIG. 9D), which resembled the AP oscillations from neonatal cardiomyocytes. Resting membrane potential was different between AP oscillation group (n=7) and non-AP oscillation group (n=20) (−61.4±3.4 mV vs −73.6±7.6 mV).

### ECG Exhibited Sustained Ventricular Beats in Adeno/_{S4T}Kv1.4_{GYS}-Treated Guinea-Pig.

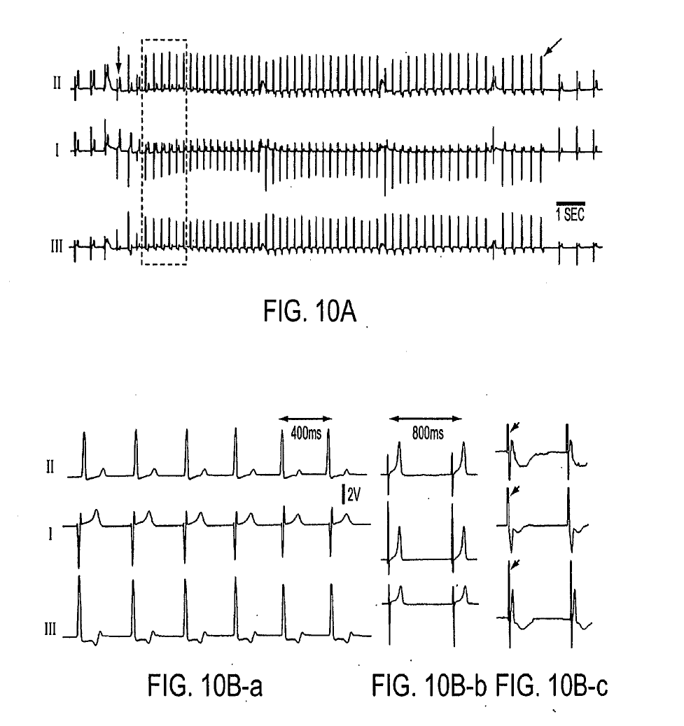

Electrocardiogram (ECG) was performed between 48 and 72 hours after virus injection. As described in materials and methods, we used methacholine (0.1-0.5 mg/g) by intra-peritoneal injection to induce bradycardia. We confirmed that methacholine did not affect _{S4T}Kv1.4_{GYS} current in HEK293 cells (data not shown). Approximately 5 minutes after methacholine injection, sinus rhythm (150 bpm) changed to complete AV-block with bradycardia (<100 bpm), and then finally to bradycardial junctional escape rhythm (<75 bpm). Control animals (Ad-GFP, n=5) showed no ectopic beats from ventricle, whereas animals injected with Ad/_{S4T}Kv1.4_{GYS} virus (n=6) showed spontaneous ventricular beats in bradycardial phase (P<0.05. versus control). In representative experiments (FIG. 10A), mapping of LV free wall with a hand-held electrode demonstrated sustained ventricular beats (150 bpm) from the virus injection site during bradycardial junctional escape rhythm. Mapping ECG (FIG. 10B-c) was not identical to ventricular beats (FIG. 10-a), but the polarities of every three leads was the same as ectopic ventricular beats from the Ad/_{S4T}Kv1.4_{GYS} virus-injected heart, indicating that electrodes were placed not exactly on the focus of ventricular beats but on peri-focus zone.

No Multimerization of SPC with HCN Gene Family.

Wild type Kv1.4 has been previously reported not to multimerize with the HCN gene family (Xue, T., Marban, E. & Li, R. A. (2002) Circ Res 90, 1267-1273.). Before in vivo use of SPC, we verified that SPC was unable to multimerize with HCN1 by co-transfection into HEK cells and analyzing reversal potentials. WtHCN1 (FIG. 3(A)-a left) expressed alone reversed at −36.1±1.4 mV, whereas HCN co-transfected with SPC exhibited a reversal potential of −22.0±8.0 mV (n=5 for each, tail currents not shown). Superfusion with Cs to block HCN1 homomultimers left behind a current which reversed at −11.1±2.3 mV, which is indistinguishable from the reversal potential of SPC alone). The clean pharmacologic separation suggests the absence of any functional SPC-HCN heteromultimers. We also excluded the possibility that SPC expression might affect native sodium, potassium, or calcium currents in adult guinea pig myocytes (data not shown).

### SPC's Pacemaker Abilities In Vivo.

Next, to test its pacemaker ability in the adult ventricle, we made bicistronic (GFP-tagged) SPC adenovirus (AdSPC) and injected it into guinea-pig heart. Seventy-two hours after virus injection, isolated ventricular myocytes transduced with AdSPC were examined by whole-cell voltage clamp. There was little measurable pacemaker current in control cells from injected animals (data not shown). In contrast, we detected hyperpolarization-activated inward current in AdSPC-transduced myocytes. Mean current densities at −80 mV or −160 mV equalled −7.2±1.3 pA/pF or −59.7±5.5 pA/pF mV, respectively (n=5 each, FIG. 3B-b). We also examined action potentials (APs) in control (n=13) and SPC-transduced cells (n=14). Control cells never exhibited spontaneous AP oscillations (SAPO), whereas half of SPC-transduced cells (seven of fourteen) showed SAPO. In the experiment shown here, we could detect fast SAPO (mean rate>200 bpm), with maximal diastolic potential (MDP) and phase 4 slope of 53.6±2.5 mV and 10.4 mV/s, respectively. There was no significant difference in evoked-AP durations (306.2±12.5 ms: control versus 303.2±10.9 ms: AdSPC-transduced cells.). Given these results, we concluded that SPC can induce pacemaker activity in guinea-pig myocytes.

To confirm the ability of SPC to induce pacemaker activity in vivo, electrocardiograms (ECG) were performed 72 hours after AdSPC injection. During ECG recording, methacholine (0.1-0.5 mg/g) was administered by intra-peritoneal injection to induce bradycardia. Control animals (AdGFP, n=6) showed no ectopic ventricular beats, whereas frequent monomorphic idioventricular beats could be detected in animals injected with AdSPC (n=6). In representative experiments, ECG with pace mapping demonstrated idioventricular rhythms (150 bpm) originating from the injection site (LV free wall). These results directly demonstrated that SPC worked as a pacemaker in vivo.

Flexibility for Frequency Tuning of SPC. Unlike previous studies with adenoviral-HCN2 delivered into other regions of the heart (Qu, J., Plotnikov, A. N., Danilo, P., Jr, Shlapakova, I., Cohen, I. S., Robinson, R. B. & Rosen, M. R. (2003) Circulation 107, 1106-1109.; Plotnikov, A. N., Sosunov, E. A., Qu, J., Shlapakova, I. N., Anyukhovsky, E. P., Liu, L., Janse, M. J., Brink, P. R., Cohen, I. S., Robinson, R. B., Danilo, P., Jr & Rosen, M. R. (2004) Circulation 109, 506-512.), we have induced biopacemaker activity with SPC in ventricular myocardium. An alternative approach has been to use mesenchymal stem cells as a platform for gene delivery to the ventricle (Potapova, I., Plotnikov, A., Lu, Z., Danilo, P., Jr, Valiunas, V., Qu, J., Doronin, S., Zuckerman, J., Shlapakova, I. N., Gao, J., Pan, Z., Herron, A. J., Robinson, R. B., Brink, P. R., Rosen, M. R. & Cohen, I. S. (2004) Circ Res 94, 952-959.). Such cells do not fully differentiate into heart cells (although they can differentiate into bone, cartilage or adipose tissue (Deans R J, M. A. (2000) Exp Hematol 28, 875-84.)), and their persistence over time has not been demonstrated. Direct gene transfer of SPC avoids many of these potential complications and uncertainties (while admittedly introducing others). Another potential advantage of SPC is its flexibility for frequency tuning of synthetic pacemaker strategy. We have investigated 3 patterns of S4 mutation and 5 kinds of pore mutation, yielding a total of possible 15 combinations of S4 and pore mutations. Some of these other mutants also expressed hyperpolarization-activated inward current in physiological conditions. For example, combining the S4 triple mutation with another pore mutation (V525S, VGYG→SGYG), has a current density of −6.1 pA/pF at −100 mV with reversal membrane potential of −25 mV in HEK cells. We also tested it in vivo and detected slow idioventricular rhythms (55 bpm) for short periods. These results indicated that specific mutations could favor specific heart rates in vivo. By combining various S4 mutations with pore mutations, we can prepare a broad range of candidates for synthetic pacemakers and choose the one best-suited to accomplish a therapeutic goal, namely pacing at any given desired basal heart rate.

In summary, by selective mutations of S4 and the pore in the human Kv1.4 channel, we succeeded in creating a novel pacemaker channel. This channel showed hyperpolarization-activated inward currents with steady activation under physiological conditions. Gene transfer of SPC induced pacemaker activity in guinea-pig adult ventricular myocardium and produced idioventricular rhythms on ECG. Given the sparse expression of Kv1 family channels in the human ventricle (Mays D J, F. J., Philipson L H, Tamkun M M (1995) J Clin Invest 96, 282-92.) and the capability of tuning the frequency of oscillation to any given desired rate range, synthetic pacemaker channels based on the Kv1 family have the potential to be novel therapeutic tools for the creation of biopacemakers.

Taken together, the above findings suggest that _{S4T}K1.4_{GYS} channels could provide synthetic pacemaker current in human myocardium. Because Kv1 family channels are sparsely-expressed in the human ventricle²⁶, we would predict that gene transfer of the synthetic pacemaker into that target tissue would be relatively uncomplicated by multimerization with endogenous subunits. Furthermore, the frequency of the resulting pacemakers could be tuned by tailoring the specific S4 mutations to the desired rate range.

### REFERENCES

The disclosure of each reference cited is expressly incorporated herein.
1. Hardy, S., Kitamura, M., Harris-Stansil, T., Dai, Y. & Phipps, M. L. Construction of adenovirus vectors through Cre-lox recombination. J Virol 71, 1842-9 (1997).
2. Hoppe, U. C., Johns, D. C., Marban, E. & O'Rourke, B. Manipulation of cellular excitability by cell fusion: effects of rapid introduction of transient outward K+ current on the guinea pig action potential. Circ Res 84, 964-72 (1999).
3. Hamill, O. P., Marty, A., Neher, E., Sakmann, B. & Sigworth, F. J. Improved patch-clamp techniques for high-resolution current recording from cells and cell-free membrane patches. Pflugers Arch 391, 85-100 (1981).
4. Neher, E. Correction for liquid junction potentials in patch clamp experiments. Methods Enzymol 207, 123-31 (1992).
5. Barry, P. H. JPCalc, a software package for calculating liquid junction potential corrections in patch-clamp, intracellular, epithelial and bilayer measurements and for correcting junction potential measurements. J Neurosci Methods 51, 107-16 (1994).
6. Zufferey, R. et al. Self-inactivating lentivirus vector for safe and efficient in vivo gene delivery. J Virol 72, 9873-80 (1998).
7. Hoppe, U. C., Marban, E. & Johns, D. C. Distinct gene-specific mechanisms of arrhythmia revealed by cardiac gene transfer of two long QT disease genes, HERG and KCNE1. Proc Natl Acad Sci USA 98, 5335-40 (2001).
8. Mitra, R. & Morad, M. Two types of calcium channels in guinea pig ventricular myocytes. Proc Natl Acad Sci USA 83, 5340-4 (1986).
9. Ennis, I. L., Li, R. A., Murphy, A. M., Marban, E. & Nuss, H. B. Dual gene therapy with SERCA1 and Kir2.1 abbreviates excitation without suppressing contractility. J Clin Invest 109, 393-400 (2002).
10. Shirahata, S., Katakura, Y. & Teruya, K. Cell hybridization, hybridomas, and human hybridomas. Methods Cell Biol 57, 111-45 (1998).
11. Lentz, B. R. & Lee, J. K. Poly(ethylene glycol) (PEG)-mediated fusion between pure lipid bilayers: a mechanism in common with viral fusion and secretory vesicle release? Mol Membr Biol 16, 279-96 (1999).
12. Gibson, A. J. et al. Dermal fibroblasts convert to a myogenic lineage in mdx mouse muscle. J Cell Sci 108 (Pt 1), 207-14 (1995).
13. Gussoni, E. et al. Long-term persistence of donor nuclei in a Duchenne muscular dystrophy patient receiving bone marrow transplantation. J Clin Invest 110, 807-14 (2002).
14. Alvarez-Dolado, M. et al. Fusion of bone-marrow-derived cells with Purkinje neurons, cardiomyocytes and hepatocytes. Nature 425, 968-73 (2003).
15. Weimann, J. M., Johansson, C. B., Trejo, A. & Blau, H. M. Stable reprogrammed heterokaryons form spontaneously in Purkinje neurons after bone marrow transplant. Nat Cell Biol 5, 959-66 (2003).
16. Potapova, I. et al. Human mesenchymal stem cells as a gene delivery system to create cardiac pacemakers. Circ Res 94, 952-9 (2004).
17. Ren, D. et al. A prokaryotic voltage-gated sodium channel. Science 294, 2372-5 (2001).
18. Sanguinetti, M. C., Jiang, C., Curran, M. E. & Keating, M. T. A mechanistic link between an inherited and an acquired cardiac arrhythmia: HERG encodes the IKr potassium channel. Cell 81, 299-307 (1995).
19. Kubo, Y., Baldwin, T. J., Jan, Y. N. & Jan, L. Y. Primary structure and functional expression of a mouse inward rectifier potassium channel. Nature 362, 127-33 (1993).
20. Azene, E. M., Xue, T., Marban, E., Tomaselli, G. F. & Li, R. A. Non-equilibrium behavior of HCN channels: insights into the role of HCN channels in native and engineered pacemakers. Cardiovasc Res 67, 263-73 (2005).
21. Qu, J. et al. Expression and function of a biological pacemaker in canine heart. Circulation 107, 1106-9 (2003).
22. Er, F. et al. Dominant-negative suppression of HCN channels markedly reduces the native pacemaker current I(f) and undermines spontaneous beating of neonatal cardiomyocytes. Circulation 107, 485-9 (2003).
23. Xue, T., Marban, E. & Li, R. A. Dominant-negative suppression of HCN1- and HCN2-encoded pacemaker currents by an engineered HCN1 construct: insights into structure-function relationships and multimerization. Circ Res 90, 1267-73 (2002).
24. Miller, A. G. & Aldrich, R. W. Conversion of a delayed rectifier K+ channel to a voltage-gated inward rectifier K+ channel by three amino acid substitutions. Neuron 16, 853-8 (1996).
25. Heginbotham, L., Lu, Z., Abramson, T. & MacKinnon, R. Mutations in the K+ channel signature sequence. Biophys J 66, 1061-7 (1994).
26. Nerbonne, J. M. Molecular basis of functional voltage-gated K⁺ channel diversity in the mammalian myocardium. J Physiol 525 Pt 2, 285-98 (2000).

I claim:

1. A method of making a heterokaryon with electrical properties from both of its parent cells, comprising:
injecting into a site in a mammal an exogenous somatic cell and a fusogen reagent, wherein the exogenous somatic cell expresses an ion channel, wherein the exogenous somatic cell fuses with an endogenous somatic cell, thereby forming a heterokaryon with electrical properties from both of its parents.

2. The method of claim 1 wherein the site is in the heart.

3. The method of claim 1 wherein the endogenous cell does not express the ion channel.

4. The method of claim 1 wherein the ion channel is a calcium channel.

5. The method of claim 1 wherein the ion channel is a Hyperpolarization-activated cyclic-nucleotide-gated (HCN) ion channel 1 (HCN1).

6. The method of claim 1 wherein the exogenous somatic cell expresses a nucleic acid sequence exogenous to it encoding the ion channel.

7. The method of claim 1 wherein the endogenous cell is a ventricular myocyte.

8. The method of claim 1 wherein the fusogen is polyethylene glycol (PEG).

9. The method of claim 8 wherein the PEG has a molecular weight of 500 to 2000.

10. The method of claim 8 wherein the PEG has a molecular weight of 1250 to 1750.

11. The method of claim 1 further comprising the step of detecting the activity of the ion channel in the heterokaryon in the mammal.

12. The method of claim 1, wherein the site of injection is a heart in the mammal, the fusogen is polyethylene glycol (PEG), and the exogenous somatic cell is an autologous or syngeneic fibroblast which expresses Hyperpolarization-activated cyclic-nucleotide-gated (HCN) ion channel 1 (HCN1) as shown in SEQ ID NO: 1 or SEQ ID NO: 5.

13. The method of claim 1 wherein the exogenous somatic cell is a fibroblast which is stably transfected with a non-viral plasmid DNA construct expressing HCN1.

14. The method of claim 1 wherein the exogenous somatic cell is a fibroblast which is stably transduced with a virus expressing HCN1.

15. The method of claim 1 wherein the exogenous cell is trypsinized prior to the step of injecting.

16. The method of claim 1 wherein the endogenous somatic cell is a neuron.

17. A method of making a biological pacemaker, comprising:
mixing myocytes, polyethylene glycol (PEG), and syngeneic or autologous fibroblasts which express Hyperpolarization-activated cyclic-nucleotide-gated (HCN) ion channel 1 (HCN1) as shown in SEQ ID NO: 1 or SEQ ID NO: 5, whereby the myocytes and the fibroblasts fuse.

18. The method of claim 17 wherein the fibroblasts are trypsinized prior to mixing.

19. The method of claim 17 wherein the PEG has a molecular weight of 500 to 2000.

20. The method of claim 17 wherein the PEG has a molecular weight of 1250 to 1750.

21. The method of claim 17 wherein the mixing is done in vitro.

22. The method of claim 17 wherein the mixing is done in vivo.

23. A method of making a biological pacemaker, comprising:
transfecting an inexcitable mammalian cell with one or more nucleic acid molecules encoding a first protein which depolarizes the cell membrane, a second protein which repolarizes the cell membrane, and a third protein which causes a cell to fire spontaneously and repetitively, whereby the mammalian cell displays spontaneously oscillating action potentials.

24. The method of claim 23 wherein the first protein is selected from the group consisting of
a voltage-dependent sodium channel, a voltage-dependent calcium channel, and a ligand-gated cation channel; the second protein is selected from the group consisting of a potassium channel and a chloride channel; and the third protein is selected from the group consisting of HCN family members.

25. The method of claim 23 wherein the one or more nucleic acid molecules are one or more plasmids.

26. The method of claim 23 wherein the mammalian cell is a human embryonic kidney cell.

27. A plasmid comprising a coding sequence for each of three ion channels, wherein said three ion channels are HCN1 (SEQ ID NO: 1 or SEQ ID NO: 5), NaChBac (SEQ ID NO: 2), and Kir2.1 (SEQ ID NO: 3 or SEQ ID NO: 6).

28. A non-naturally occurring voltage-dependent K⁺ channel protein which activates upon hyperpolarization and is non-selective to monovalent cations.

29. A nucleic acid encoding the channel protein according to claim 28.

30. A nucleic acid vector which comprises the nucleic acid of claim 29.

31. The nucleic acid vector of claim 30 which is a virus vector.

32. A method of administering a nucleic acid vector according to claim 31, comprising:
injecting the virus into a mammal.

33. A hyperpolarization-activated, inward current, channel protein comprising four mutations relative to wild-type sequence of a Kv1.4 protein according to SEQ ID NO: 4, wherein said four mutations are R447N, L448A, R453I, and G528S.

34. A nucleic acid encoding the hyperpolarization-activated inward current channel protein according to claim 33.

35. A nucleic acid vector which comprises the nucleic acid of claim 34.

36. The nucleic acid vector of claim 35 which is a virus vector.

37. A method of administering an nucleic acid vector according to claim 36, comprising:
injecting the virus into a mammalian heart.

38. The method of claim 37 wherein the virus is injected into an atrium of the mammalian heart.

39. The method of claim 37 wherein the virus is injected into a left ventricle of the mammalian heart.


SEQUENCE LISTING (6 sequences)

SEQ ID NO: 1 - protein, 910 aa, Mus musculus
MEGGGKPNSASNSRDDGNSVFPSKAPATGPVAADKRLGTPPRGGAAGKEHGNSVCFKVDGGGGEEPAGSFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILSELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIPPINYPQMTALNCTSSTTTPTSRMRTQSPPVYTATSLSHSNLHSPSPSTQTPQPSAILSPCSYTTAVCSPPIQSPLATRTFHYASPTASQLSLMQQPQQQLPQSQVQQTQTQTQQQQQQQQQQQQQQQQQQQQQQQQQQQQQQQQQQQQQPQTPGSSTPKNEVHKSTQALHNTNLTKEVRPLSASQPSLPHEVSTLISRPHPTVGESLASIPQPVAAVHSTGLQAGSRSTVPQRVTLFRQMSSGAIPPNRGVPPAPPPPAAVQRESPSVLNTDPDAEKPRFASNL

SEQ ID NO: 2 - protein, 284 aa, Bacillus pseudofirmus
MENNPAEQQVPPLVALAQRIVFHKAFTPTIITLIIINAIIVGLETYPTVYQGYNDWFYAADLALLWIFTIEITLRFIAARPTKSFFKSSWNWFDLLIVLAGHVFAGAHFVTVLRILRVLRVLRAISVIPSLRRLVDALLMTIPALGNIMILMGIIFYIFAVIGTMLFASVAPEYFGNLQLSLLTLFQVVTLESWASGVMRPIFAEVWWSWIYFVIFILVGTFIVFNLFIGVIVNNVEKANEEELKSELDDKEADTKEELASLRNEVAEMKDLIKQMHKQQTKKG

SEQ ID NO: 3 - protein, 428 aa, Mus musculus [sequence omitted]

SEQ ID NO: 4 - protein, 653 aa, Homo sapiens
MEVAMVSAESSGCNSHMPYGYAAQARARERERLAHSRAAAAAAVAAATAAVEGSGGSGGGSHHHHQSRGACTSHDPQSSRGSRRRRRQRSEKKKAHYRQSSFPHCSDLMPSGSEEKILRELSEEEEDEEEEEEEEEEGRFYYSEDDHGDECSYTDLLPQDEGGGGYSSVRYSDCCERVVINVSGLRFETQMKTLAQFPETLLGDPEKRTQYFDPLRNEYFFDRNRPSFDAILYYYQSGGRLKRPVNVPFDIFTEEVKFYQLGEEALLKFREDEGFVREEEDRALPENEFKKQIWLLFEYPESSSPARGIAIVSVLVILISIVIFCLETLPEFRDDRDLVMALSAGGHGGLLNDTSAPHLENSGHTIFNDPFFIVETVCIVWFSFEFVVRCFACPSQALFFKNIMNIIDIVSILPYFITLGTDLAQQQGGGNGQQQQAMSFAILRIIRLVRVFRIFKLSRHSKGLQILGHTLRASMRELGLLIFFLFIGVILFSSAVYFAEADEPTTHFQSIPDAFWWAVVTMTTVGYGDMKPITVGGKIVGSLCAIAGVLTIALPVPVIVSNFNYFYHRETENEEQTQLTQNAVSCPYLPSNLLKKFRSSTSSSLGDKSEYLEMEEGVKESLCAKEEKCQGKGDDSETDKNNCSNAKAVETDV

SEQ ID NO: 5 - protein, 890 aa, Homo sapiens
MEGGGKPNSSSNSRDDGNSVFPAKASATGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWSVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLRGEIVNFNCRKLVATMPLFANADPNFVTAMLSKLRFEVFQPGDYIVREGAVGKKMYFIQHGVAGVITKSSKEMKLTDGSYFGEICLLTKGRRTASVRADTYCRLYSLSVDNFNEVPEEYPMMRRAFETVAIDRLDRIGKKNSILLQKFQKDLNTGVFNNQENEILKQIVKHDREMVQAIAPINYPQMTTLNSASSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPLSASQPSLPHEVPTLISRPHPTVGESLASIPQPVTAVPGTGLQAGGRSTVPQRVTLFRQMSSGAIPPNRGVPPAPPPPAAALPRESSSVLNTDPDAEKPRFASNL

SEQ ID NO: 6 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI